(19) United States
(12) Patent Application Publication
(10) Pub. No.: US 2022/0135566 A1
(43) Pub. Date: May 5, 2022

(54) CRYSTALLINE SOLID FORMS OF BARICITINIB

(71) Applicant: TEVA PHARMACEUTICALS INTERNATIONAL GMBH, Jona (CH)

(72) Inventors: Ivana Landeka, Zagreb (HR); Dijana Skalec Samec, Jastrebarsko (HR); Lorena Kordic, Rab (HR)

(21) Appl. No.: 17/427,958

(22) PCT Filed: Feb. 5, 2020
(86) PCT No.: PCT/US20/16721

§ 371 Date: Aug. 3, 2021

Related U.S. Application Data
(60) Provisional application No. 62/816,256, filed on Mar. 11, 2019.
(60) Provisional application No. 62/801,161, filed on Feb. 5, 2019.

(51) Int. Cl. C07D 487/04

(52) U.S. Cl. CPC C07D 487/04; C07B 2200/13

(57) ABSTRACT

The present disclosure relates to cocrystals/salts of baricitinib, processes for preparation thereof as well as a pharmaceutical composition comprising the same.

### TECHNICAL FIELD

The present disclosure relates to cocrystals/salts of baricitinib, processes for preparation thereof as well as a pharmaceutical composition comprising the same.

### BACKGROUND

Baricitinib has the chemical name (1-(ethylsulfonyl)-3-[4-(7H-pyrrolo[2,3-d]pyrimidin-4-yl)-1H-pyrazol-1-yl]-3-azetidineacetonitrile. Baricitinib has the following chemical structure:

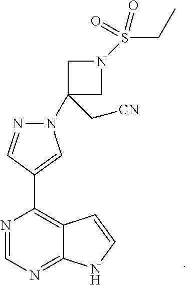 [SMILES: CCS(=O)(=O)N1CC(CC#N)(n2cc(-c3ncnc4[nH]ccc34)cn2)C1]

Baricitinib is a JAK inhibitor indicated for the treatment of adult patients with moderately to severely active rheumatoid arthritis who have had an inadequate response to one or more TNF antagonist therapies.

Baricitinib is disclosed in U.S. Pat. No. 8,158,616.

Crystalline forms of baricitinib and salts thereof are disclosed in WO2015/166434, WO2016/141891, IPCOM000244270D, CN 105693731, WO2019/003249, WO2018/113801, WO2018/099680, CZ31155, CN105566332, CN105601635, TW1616447, EP3321267 and WO2018/233437.

Polymorphism, the occurrence of different crystal forms, is a property of some molecules and molecular complexes. A single compound, like baricitinib or salt thereof, may give rise to a variety of polymorphs having distinct crystal structures and physical properties like melting point, thermal behaviors (e.g. measured by thermogravimetric analysis—“TGA”, or differential scanning calorimetry—“DSC”), powder X-ray diffraction (PXRD) pattern, infrared absorption fingerprint, Raman absorption fingerprint, and solid state (¹³C-) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms, cocrystals and solvates of a pharmaceutical product can provide materials having desirable processing properties, such as ease of handling, ease of processing, storage stability, and ease of purification or as desirable intermediate crystal forms that facilitate conversion to other salts or polymorphic forms. New salts, polymorphic forms, cocrystals and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

In the case of baricitinib, many salts are known but there is a need for a solid crystalline form which offers superior physico-chemical properties (such as stability, solubility) without altering the pharmacological properties.

### SUMMARY

The present disclosure relates to cocrystals of baricitinib, in particular to cocrystal forms of baricitinib and orotic acid, cocrystal forms of baricitinib and naphthalene-2-sulfonic acid, cocrystal forms of baricitinib and (+)-camphoric acid, cocrystal forms of baricitinib and fumaric acid, cocrystal forms of baricitinib and tartaric acid, cocrystal forms of baricitinib and succinic acid, to solid state forms thereof, to processes for preparation thereof, and to pharmaceutical compositions including these solid state forms or combinations thereof.

The present disclosure encompasses process for preparation of cocrystal forms of baricitinib and solid state forms thereof including reacting baricitinib with a co-former in a molar ratio of between about 1:1 to about 1:2, in embodiments in a molar ratio of about 1:1.

The present disclosure also provides uses of the cocrystals of baricitinib and solid state forms thereof for preparing other solid state forms of baricitinib, salts of baricitinib and solid state forms thereof.

In another embodiment, the present disclosure encompasses the above described cocrystals of baricitinib and solid state forms thereof for use in the preparation of pharmaceutical compositions and/or formulations, in embodiments for the treatment of rheumatoid arthritis.

In another embodiment the present disclosure encompasses the use of the above described cocrystals of baricitinib and solid state forms thereof for the preparation of pharmaceutical compositions and/or formulations.

The present disclosure further provides pharmaceutical compositions including cocrystals of baricitinib and solid state forms thereof.

In yet another embodiment, the present disclosure encompasses pharmaceutical formulations including cocrystals of baricitinib and solid state forms thereof or combinations thereof and at least one pharmaceutically acceptable excipient. The pharmaceutical composition or formulation includes oral dosage forms, e.g. tablet or capsule. The present disclosure encompasses processes to prepare said pharmaceutical formulations of cocrystals of baricitinib and solid state forms thereof including combining cocrystals of baricitinib and solid state forms thereof or combinations thereof, prepared according to the present disclosure with at least one pharmaceutically acceptable excipient.

The cocrystals of baricitinib and solid state forms thereof as defined herein, as well as the pharmaceutical compositions or formulations of cocrystals of baricitinib and solid state forms thereof prepared according to the present disclosure, can be used as medicaments, particularly for the treatment of rheumatoid arthritis.

The present disclosure also provides methods of treating rheumatoid arthritis by administering a therapeutically effective amount of cocrystals of baricitinib and solid state forms thereof or combinations thereof prepared according to the present disclosure, or at least one of the above pharmaceutical compositions or formulations, to a subject suffering from rheumatoid arthritis, or otherwise in need of the treatment.

The present disclosure also provides uses of cocrystals of baricitinib and solid state forms thereof of the present disclosure, or at least one of the above pharmaceutical compositions or formulations for the manufacture of a medicament for treating rheumatoid arthritis.

### BRIEF DESCRIPTION OF THE FIGURES

FIG. 1 shows a powder X-ray diffraction pattern (“powder XRD” or “PXRD”) of a cocrystal of Baricitinib and orotic acid form I;

FIG. 2 shows a PXRD of cocrystal of Baricitinib and orotic acid form II;

FIG. 3 shows a PXRD of Baricitinib naphthalene-2-sulfonate form I;

FIG. 4 shows a PXRD of cocrystal of Baricitinib and (+)-camphoric acid form I;

FIG. 5 shows a PXRD of cocrystal of Baricitinib and orotic acid form IV;

FIG. 6 shows a PXRD of cocrystal of Baricitinib and fumaric acid form I;

FIG. 7 shows a PXRD of cocrystal of Baricitinib and tartaric acid form I;

FIG. 8 shows a PXRD of cocrystal of Baricitinib and tartaric acid form II;

FIG. 9 shows a PXRD of cocrystal of Baricitinib and succinic acid form I;

FIG. 10 shows a 1H-13C CP-MAS spectra of Baricitinib and L-(+)-tartaric acid co-crystal Form I; and

FIG. 11 shows 1H-13C CP-MAS spectra of Baricitinib and fumaric acid co-crystal Form I.

### DETAILED DESCRIPTION

The present disclosure relates to cocrystals of baricitinib and a cocrystal former and solid state forms thereof, to salts of baricitinib and solid state forms thereof, to processes for preparation thereof, and to pharmaceutical compositions including these solid state forms or combinations thereof.

The cocrystals of baricitinib and solid state forms thereof according to the present disclosure may have advantageous properties selected from at least one of: chemical or polymorphic purity, flowability, solubility, dissolution rate, bioavailability, morphology or crystal habit, stability such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of baricitinib referred to herein as being characterized by graphical data “as depicted in” a Figure will thus be understood to include any crystal forms of the baricitinib, characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or about 0% of any other forms of the subject compound as measured, for example, by PXRD. Thus, cocrystal of baricitinib or solid state forms thereof described herein as substantially free of any other solid state forms would be understood to contain greater than about 80% (w/w), greater than about 90% (w/w), greater than about 95% (w/w), greater than about 98% (w/w), greater than about 99% (w/w), or about 100% (w/w) of the subject cocrystal of baricitinib and solid state forms thereof. Accordingly, in some embodiments of the disclosure, the described cocrystal of baricitinib and solid state forms thereof may contain from about 1% to about 20% (w/w), from about 5% to about 20% (w/w), or from about 5% to about 10% (w/w) of one or more other solid state forms of the same baricitinib.

“Co-Crystal” or “Cocrystal” as used herein is defined as a crystalline material including two or more molecules in the same crystalline lattice and associated by non-ionic and non-covalent bonds. In some embodiments, the cocrystal includes two molecules which are in natural state.

“Cocrystal former” or “crystal former” as used herein is defined as a molecule that forms a cocrystal with baricitinib, for example orotic acid and/or naphthalene-2-sulfonic acid.

The modifier “about” should be considered as disclosing the range defined by the absolute values of the two endpoints. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9-1.1.

As used herein, unless stated otherwise, PXRD peaks reported herein are preferably measured using CuK_{α} radiation, λ=1.5418 Å.

As used herein, unless stated otherwise, ¹³C NMR reported herein are measured at 150 MHz at a magic angle spinning frequency ωᵣ/2π=16 kHz, preferably at a temperature of at 293 K±3° C. As used herein, unless stated otherwise, single crystal data reported herein are preferably measured using graphite-monochromated MoKα (λ=0.71073 Å) radiation at the using the ω scan mode over the 20 range up to 54°, at 150 Kelvin.

As used herein, and unless stated otherwise, the term “anhydrous” in relation to crystalline forms of baricitinib (and cocrystals thereof), relates to a crystalline form of baricitinib (and cocrystals thereof) which does not include any crystalline water (or other solvents) in a defined, stoichiometric amount within the crystal. Moreover, an “anhydrous” form would generally not contain more than 1% (w/w), of either water or organic solvents as measured for example by TGA.

As used herein, the term “isolated” in reference to solid state forms of a cocrystal with baricitinib or salts of baricitinib of the present disclosure corresponds to solid state forms of a cocrystal with baricitinib/salts of baricitinib that are physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

As used herein, the expression “wet crystalline form” refers to a polymorph that was not dried using any conventional techniques to remove residual solvent. Examples for such conventional techniques can be, but not limited to, evaporation, vacuum drying, oven drying, drying under nitrogen flow, etc.

As used herein, the expression “dry crystalline form” refers to a polymorph that was dried using any conventional techniques to remove residual solvent. Examples of such conventional techniques can be, but are not limited to, evaporation, vacuum drying, oven drying, drying under nitrogen flow, etc.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

The present disclosure includes cocrystals of baricitinib and orotic acid (baricitinib:orotic acid).

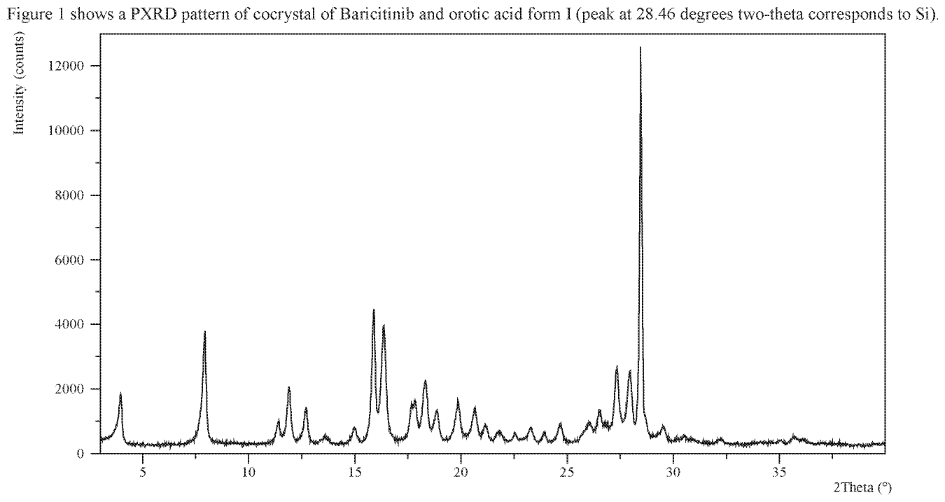

The present disclosure further includes a cocrystal of baricitinib and orotic acid designated as Form I. Form I of baricitinib:orotic acid cocrystal can be characterized by data selected from one or more of the following: a PXRD pattern having peaks at 7.9, 11.9, 15.9, 18.3 and 27.3 degrees 2-theta±0.2 degrees 2-theta; a PXRD pattern as depicted in FIG. 1; or combinations of these data.

Form I of baricitinib:orotic acid cocrystal may be further characterized by the PXRD pattern having peaks at 7.9, 11.9, 15.9, 18.3 and 27.3 degrees 2-theta±0.2 degrees 2-theta, and also having one, two, three, four or five additional peaks at 4.0, 12.7, 16.3, 19.9 and 24.8 degrees 2-theta±0.2 degrees 2-theta.

Form I of baricitinib:orotic acid cocrystal may be characterized by each of the above characteristics alone/or by all possible combinations, e.g., by PXRD pattern having peaks at 7.9, 11.9, 15.9, 18.3 and 27.3 degrees 2-theta±0.2 degrees 2-theta and a PXRD pattern as depicted in FIG. 1.

Form I of baricitinib:orotic acid cocrystal according to any of the above embodiments may be in a molar ratio between about 1:1.5 and 1.5:1, between about 1:1.25 and 1.25:1, in another embodiment in a molar ratio of about 1:1.

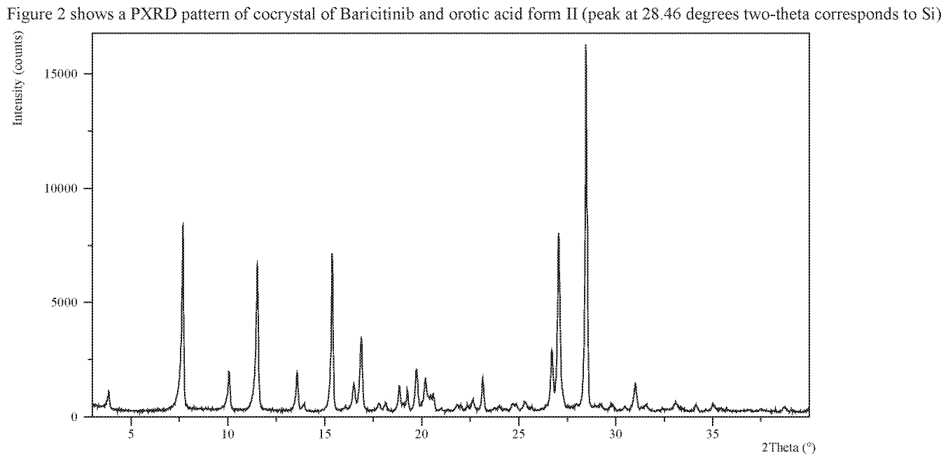

The present disclosure further includes a cocrystal of baricitinib and orotic acid designated as Form II. Form II of baricitinib:orotic acid cocrystal can be characterized by data selected from one or more of the following: a PXRD pattern having peaks at 7.7, 10.1, 11.5 and 15.4 degrees 2-theta±0.2 degrees 2-theta; a PXRD pattern as depicted in FIG. 2; or combinations of these data.

Form II of baricitinib:orotic acid cocrystal may be further characterized by the PXRD pattern having peaks at 7.7, 10.1, 11.5 and 15.4 degrees 2-theta±0.2 degrees 2-theta, and also having one, two, three or four additional peaks at 13.6, 16.9, 18.8 and 23.1 degrees 2-theta±0.2 degrees 2-theta.

Form II of baricitinib:orotic acid cocrystal may be characterized by each of the above characteristics alone/or by all possible combinations, e.g., by PXRD pattern having peaks at 7.7, 10.1, 11.5 and 15.4 degrees 2-theta±0.2 degrees 2-theta and a PXRD pattern as depicted in FIG. 2.

Form II of baricitinib:orotic acid cocrystal according to any of the above embodiments may be in a molar ratio between about 1:1.5 and 1.5:1, between about 1:1.25 and 1.25:1, in another embodiment in a molar ratio of about 1:1.

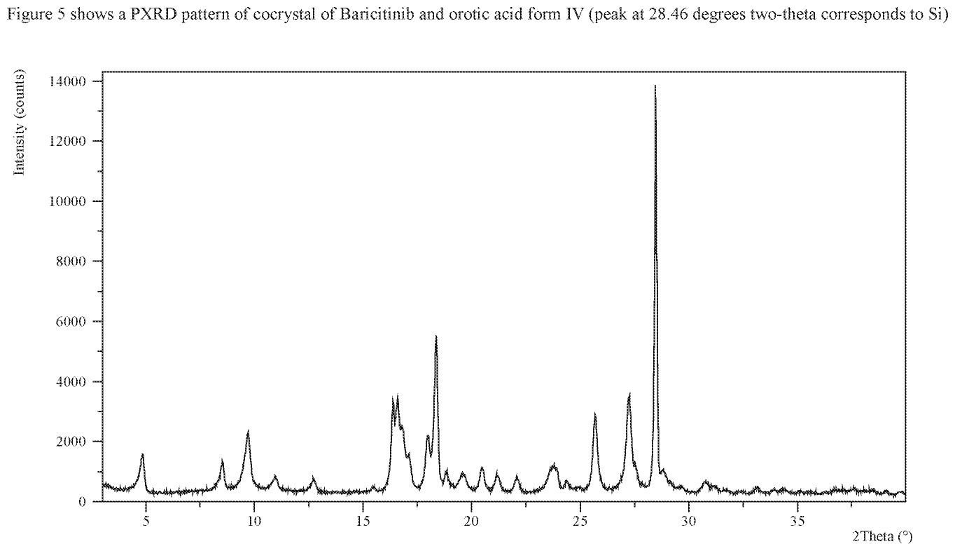

The present disclosure further includes a cocrystal of baricitinib and orotic acid designated as Form IV. Form IV of baricitinib:orotic acid cocrystal can be characterized by data selected from one or more of the following: a PXRD pattern having peaks at 4.9, 8.5, 9.7, 20.5 and 25.7 degrees 2-theta±0.2 degrees 2-theta; a PXRD pattern as depicted in FIG. 5; or combinations of these data.

Form IV of baricitinib:orotic acid cocrystal may be further characterized by the PXRD pattern having peaks at 4.9, 8.5, 9.7, 20.5 and 25.7 degrees 2-theta±0.2 degrees 2-theta, and also having one, two, three or four additional peaks at 10.9, 16.6, 18.0, 18.4 and 27.3 degrees 2-theta±0.2 degrees 2-theta.

Form IV of baricitinib:orotic acid cocrystal may be characterized by each of the above characteristics alone/or by all possible combinations, e.g., by a PXRD pattern having peaks at 4.9, 8.5, 9.7, 20.5 and 25.7 degrees 2-theta±0.2 degrees 2-theta and a PXRD pattern as depicted in FIG. 5.

Form IV of baricitinib:orotic acid cocrystal according to any of the above embodiments may be in a molar ratio between about 1:1.5 and 1.5:1, between about 1:1.25 and 1.25:1, in another embodiment in a molar ratio of about 1:1.

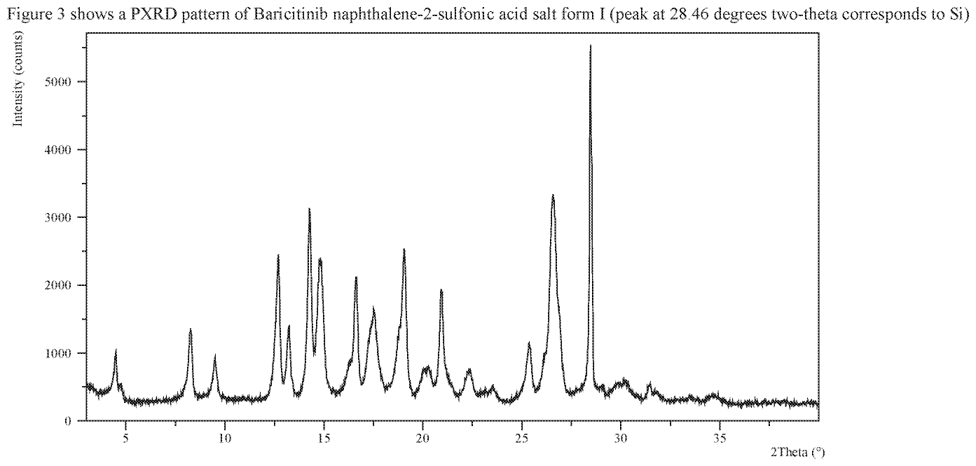

The present disclosure includes a salt of baricitinib and naphthalene-2-sulfonic acid (baricitinib naphthalene-2-sulfonate) designated as Form I. Form I of baricitinib naphthalene-2-sulfonate can be characterized by data selected from one or more of the following: a PXRD pattern having peaks at 8.3, 9.5, 14.3, 14.8 and 20.9 degrees 2-theta±0.2 degrees 2-theta; a PXRD pattern as depicted in FIG. 3; or combinations of these data.

Form I of baricitinib naphthalene-2-sulfonate may be further characterized by the PXRD pattern having peaks at 8.3, 9.5, 14.3, 14.8 and 20.9 degrees 2-theta±0.2 degrees 2-theta, and also having one, two, three, four or five additional peaks at 4.5, 12.7, 13.2, 16.7 and 19.1 degrees 2-theta±0.2 degrees 2-theta.

Form I of baricitinib naphthalene-2-sulfonate may be characterized by each of the above characteristics alone/or by all possible combinations, e.g., by PXRD pattern having peaks at 8.3, 9.5, 14.3, 14.8 and 20.9 degrees 2-theta±0.2 degrees 2-theta and a PXRD pattern as depicted in FIG. 3.

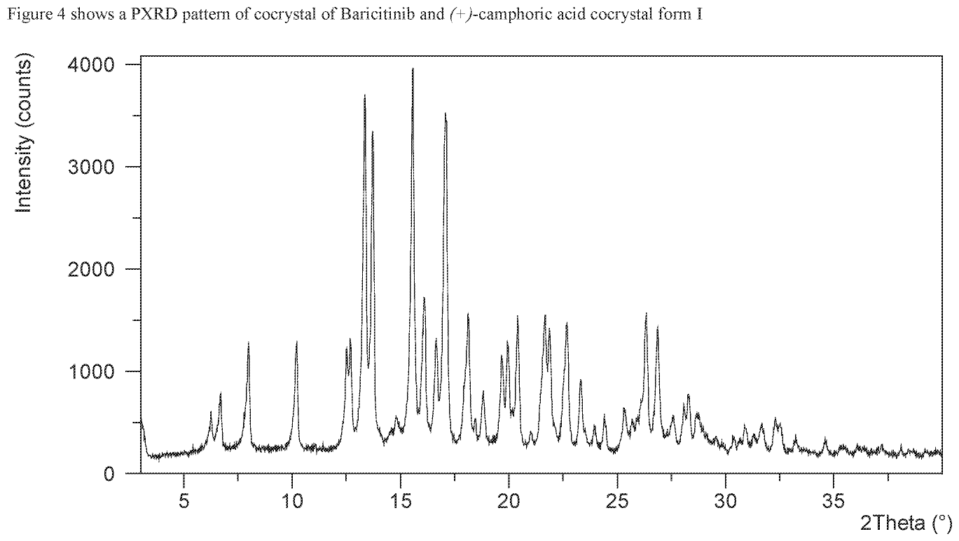

The present disclosure further includes a cocrystal of baricitinib and (+)-camphoric acid (baricitinib:(+)-camphoric acid) designated as Form I. Form I of baricitinib:(+)-camphoric acid cocrystal can be characterized by data selected from one or more of the following: a PXRD pattern having peaks at 8.0, 10.2, 13.4, 15.6 and 17.1 degrees 2-theta±0.2 degrees 2-theta; a PXRD pattern as depicted in FIG. 4; or combinations of these data.

Form I of baricitinib:(+)-camphoric acid cocrystal may be further characterized by the PXRD pattern having peaks at 8.0, 10.2, 13.4, 15.6 and 17.1 degrees 2-theta±0.2 degrees 2-theta, and also having one, two, three, four or five additional peaks at 6.7, 13.7, 16.1, 18.1 and 21.7 degrees 2-theta±0.2 degrees 2-theta.

Form I of baricitinib:(+)-camphoric acid cocrystal may be characterized by each of the above characteristics alone/or by all possible combinations, e.g., by PXRD pattern having peaks at 8.0, 10.2, 13.4, 15.6 and 17.1 degrees 2-theta±0.2 degrees 2-theta and a PXRD pattern as depicted in FIG. 4.

Form I of baricitinib:(+)-camphoric acid cocrystal may be in a molar ratio between about 1:1.5 and 1.5:1, between about 1:1.25 and 1.25:1, in another embodiment a molar ratio of about 1:1.

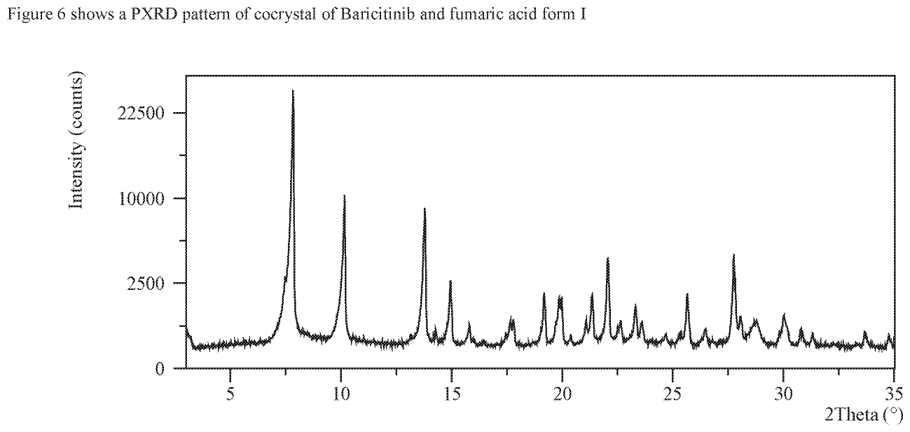

The present disclosure further includes a cocrystal of baricitinib and fumaric acid (baricitinib:fumaric acid) designated as Form I. Form I of baricitinib:fumaric acid cocrystal may be characterized by data selected from one or more of the following: a PXRD pattern having peaks at 7.9, 10.2, 13.8, 15.0 and 23.6 degrees 2-theta±0.2 degrees 2-theta; a PXRD pattern as depicted in FIG. 6; or combinations of these data.

Form I of baricitinib:fumaric acid cocrystal may be further characterized by a PXRD pattern having peaks at 7.9, 10.2, 13.8, 15.0 and 23.6 degrees 2-theta±0.2 degrees 2-theta, and also having one, two, three, four or five additional peaks at 17.8, 19.2, 22.1, 23.3 and 25.7 degrees 2-theta±0.2 degrees 2-theta.

Form I of baricitinib:fumaric acid cocrystal may be alternatively characterized by a PXRD pattern having peaks at: 7.9, 10.2, 13.8, 15.0, 17.8, 19.2, 22.1, 23.3, 23.6, and 25.7 degrees 2-theta±0.2 degrees 2-theta.

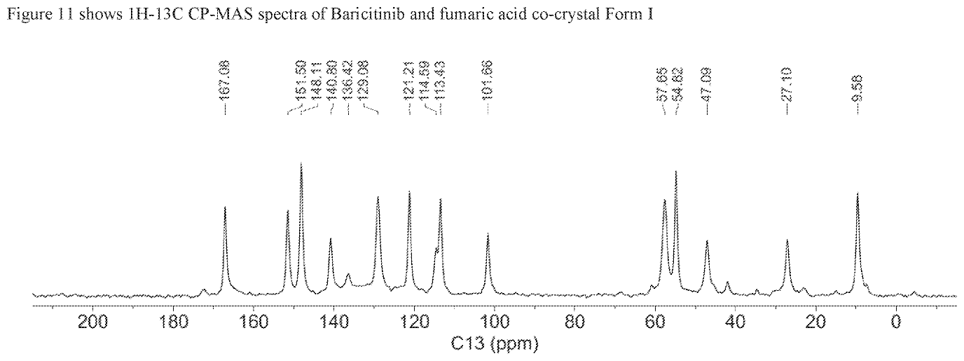

In any embodiment of the present invention Form I of baricitinib:fumaric acid cocrystal may alternatively or additionally be characterized by a solid state 13C NMR spectrum having peaks at 148.1, 129.1, 121.2, 101.7±0.2 ppm. Form I of baricitinib:fumaric acid cocrystal may alternatively or additionally be characterized by a solid state 13C NMR spectrum having the following chemical shift absolute differences from a reference peak at 9.6 ppm±0.2 ppm of 138.5, 119.5, 111.6 and 92.1±0.1 ppm. In any embodiment of the present invention, Form I of baricitinib:fumaric acid cocrystal may alternatively or additionally be characterized by a solid state 13C NMR spectrum substantially as depicted in FIG. 11.

Form I of baricitinib:fumaric acid cocrystal according to any embodiment of the invention may be in a molar ratio between about 1:0.3 to about 1:1; about 1:0.4 to about 1:0.6, or about 1:0.5.

Form I of baricitinib:fumaric acid cocrystal according to any embodiment of the invention may be an anhydrous form.

Form I of baricitinib:fumaric acid cocrystal may alternatively or additionally be characterized by unit cell parameters substantially as follows:
Cell lengths: a=6.7554(9) Å, b=12.8893(13) Å, c=23.925(3) Å
Cell angles: α=90, β=97.528(13)°, γ=90°
Cell volume: 2065.25 Å³Space group P2₁/c
Z: 4

Form I of baricitinib:fumaric acid cocrystal may be characterized by each of the above characteristics alone/or by all possible combinations, e.g., by PXRD pattern having peaks at 7.9, 10.2, 13.8, 15.0 and 23.6 degrees 2-theta±0.2 degrees 2-theta and a PXRD pattern as depicted in FIG. 6.

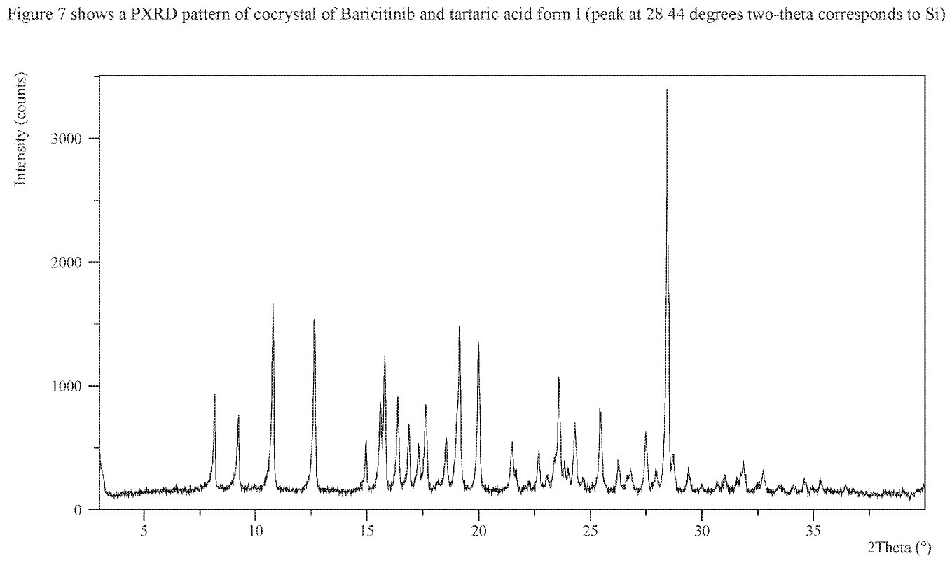

The present disclosure further includes a cocrystal of baricitinib and tartaric acid (baricitinib:tartaric acid) designated as Form I. Form I of baricitinib:tartaric acid cocrystal may be characterized by data selected from one or more of the following: a PXRD pattern having peaks at 16.4, 15.6, 10.8, 9.3 and 8.2 degrees 2-theta±0.2 degrees 2-theta; a PXRD pattern as depicted in FIG. 7; or combinations of these data.

Form I of baricitinib:tartaric acid cocrystal may be further characterized by a PXRD pattern having peaks at 16.4, 15.6, 10.8, 9.3 and 8.2 degrees 2-theta±0.2 degrees 2-theta, and also having one, two, three, four or five additional peaks at 24.3, 21.5, 17.6, 16.9 and 15.8 degrees 2-theta±0.2 degrees 2-theta.

Form I of baricitinib:tartaric acid cocrystal may be alternatively characterized by a PXRD pattern having peaks at 24.3, 21.5, 17.6, 16.9, 16.4, 15.8, 15.6, 10.8, 9.3 and 8.2 degrees 2-theta±0.2 degrees 2-theta.

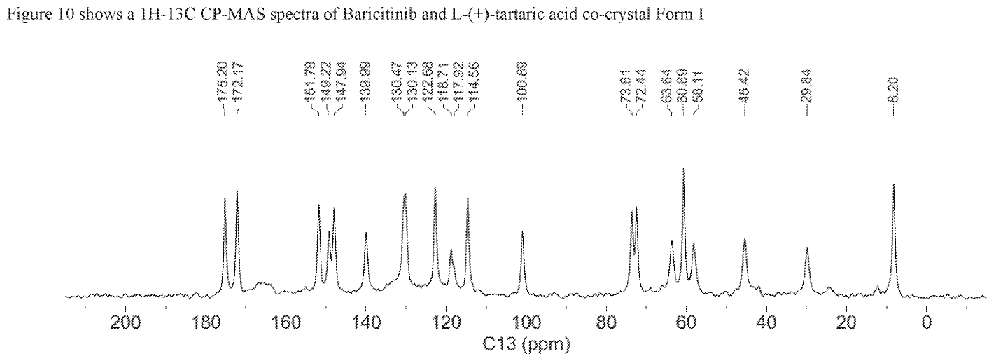

In any embodiment of the present invention, Form I of baricitinib:tartaric acid cocrystal may alternatively or additionally be characterized by a solid state ¹³C NMR spectrum having peaks at 147.9, 140.0, 122.7, 114.6, 8.2±0.2 ppm. Form I of baricitinib:fumaric acid cocrystal may alternatively or additionally be characterized by a solid state ¹³C NMR spectrum having the following chemical shift absolute differences from a reference peak at 8.2 ppm±0.2 ppm of 139.7, 131.8, 114.5 and 106.4±0.1 ppm±0.1 ppm. In any embodiment of the present invention, Form I of baricitinib:fumaric acid cocrystal may alternatively or additionally be characterized by a solid state 13C NMR spectrum substantially as depicted in FIG. 10.

In any embodiment of the present invention, the tartaric acid is preferably L-(+)-tartaric acid. Thus, in a preferred embodiments the present invention provides a cocrystal as defined in any of the above embodiments, which is a co-crystal Form I of baricitinib:L-(+)-tartaric acid.

Form I of baricitinib:tartaric acid cocrystal according to any embodiment of the invention may be in a molar ratio between about 1:0.5 to about 1:2, or about 1:0.8 to about 1:1.1, or about 1:1.

In any embodiment of the present invention, the tartaric acid in the Form I baricitinib:tartaric acid cocrystal is L-(+)-tartaric acid.

Form I of baricitinib:tartaric acid cocrystal according to any embodiment of the invention may be an anhydrous form.

Form I of baricitinib:tartaric acid cocrystal may alternatively or additionally be characterized by unit cell parameters substantially as follows:

Cell lengths: a=5.6685(2) Å, b=11.3619(4) Å, c=35.5065(15) Å;

Cell angles: α=90°, β=90°, γ=90°;

Cell volume: 2286.79 Å³; and;

Space group: P2₁2₁2₁

Z: 4

Form I of baricitinib:tartaric acid cocrystal may be characterized by each of the above characteristics alone/or by all possible combinations, e.g., by PXRD pattern having peaks at 16.4, 15.6, 10.8, 9.3 and 8.2 degrees 2-theta±0.2 degrees 2-theta and a PXRD pattern as depicted in FIG. 7.

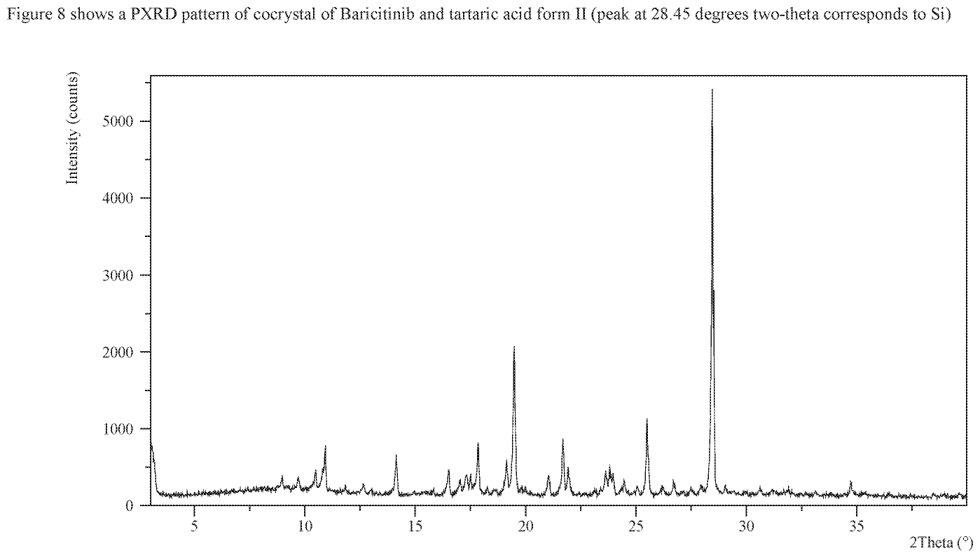

The present disclosure further includes a cocrystal of baricitinib and tartaric acid (baricitinib:tartaric acid) designated as Form II. Form II of baricitinib:tartaric acid cocrystal may be characterized by data selected from one or more of the following: a PXRD pattern having peaks at 19.5, 17.8, 14.1, 9.7 and 9.0 degrees 2-theta±0.2 degrees 2-theta; a PXRD pattern as depicted in FIG. 8; or combinations of these data.

Form II of baricitinib:tartaric acid cocrystal may be further characterized by a PXRD pattern having peaks at 19.5, 17.8, 14.1, 9.7 and 9.0 degrees 2-theta±0.2 degrees 2-theta, and also having one, two, three, four or five additional peaks at 21.9, 21.0, 16.5, 11.8 and 10.5 degrees 2-theta±0.2 degrees 2-theta.

Form II of baricitinib:tartaric acid cocrystal may be alternatively characterized by a PXRD pattern having peaks at 21.9, 21.0, 19.5, 17.8, 16.5, 14.1, 11.8, 10.5, 9.7 and 9.0 degrees 2-theta±0.2 degrees 2-theta.

Form II of baricitinib:tartaric acid cocrystal may be characterized by each of the above characteristics alone/or by all possible combinations, e.g., by PXRD pattern having peaks at 19.5, 17.8, 14.1, 9.7 and 9.0 degrees 2-theta±0.2 degrees 2-theta and a PXRD pattern as depicted in FIG. 8.

In any embodiment of the present invention, the tartaric acid in the Form II baricitinib:tartaric acid cocrystal is L-(+)-tartaric acid.

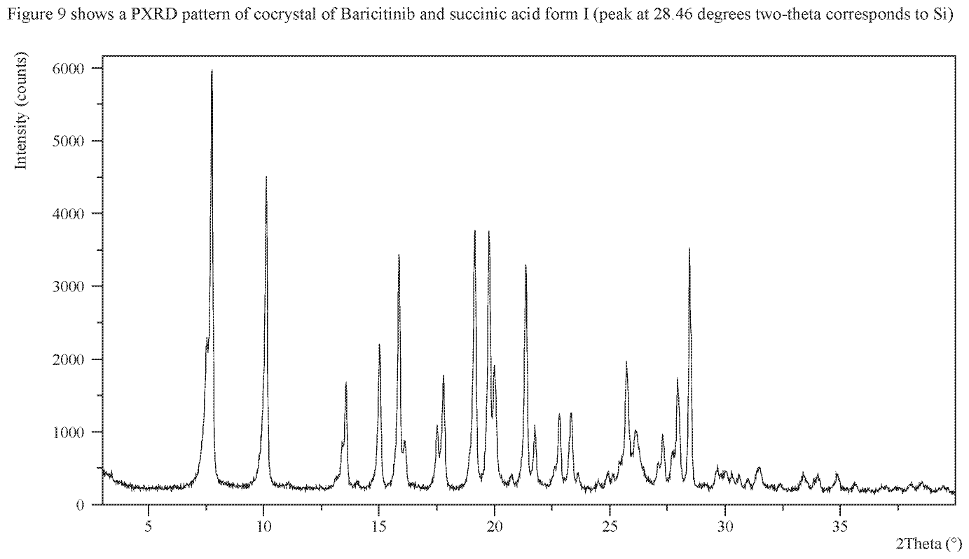

The present disclosure further includes a cocrystal of baricitinib and succinic acid (baricitinib: succinic acid) designated as Form I. Form I of baricitinib: succinic acid cocrystal may be characterized by data selected from one or more of the following: a PXRD pattern having peaks at 21.4, 17.8, 13.6, 10.1 and 7.7 degrees 2-theta±0.2 degrees 2-theta; a PXRD pattern as depicted in FIG. 9; or combinations of these data.

Form I of baricitinib: succinic acid cocrystal may be further characterized by a PXRD pattern having peaks at 21.4, 17.8, 13.6, 10.1 and 7.7 degrees 2-theta±0.2 degrees 2-theta, and also having one, two, three, four or five additional peaks at 27.9, 23.3, 22.8, 19.8 and 15.9 degrees 2-theta±0.2 degrees 2-theta.

Form I of baricitinib: succinic acid cocrystal may be characterized by each of the above characteristics alone/or by all possible combinations, e.g., by PXRD pattern having peaks at 21.4, 17.8, 13.6, 10.1 and 7.7 degrees 2-theta±0.2 degrees 2-theta and a PXRD pattern as depicted in FIG. 9.

The present disclosure also provides the use of the cocrystals/salts of baricitinib and solid state forms thereof of the present disclosure for preparing different solid state forms of baricitinib, salts of baricitinib and solid state forms thereof.

The present disclosure further encompasses processes for preparing the cocrystals/salts of baricitinib and solid state forms thereof of the present disclosure. The disclosure further includes processes for preparing different cocrystals/salts and solid state forms of baricitinib or salts of baricitinib and solid state forms thereof. The process includes preparing at least one of the cocrystals/salts of baricitinib and solid state forms thereof of the present disclosure, and converting it to different solid state forms of baricitinib or salts of baricitinib and solid state forms thereof. The conversion can be done, for example, by a process including reacting at least one of the obtained cocrystals/salts of baricitinib with an appropriate acid to obtain baricitinib acid addition salt.

In another embodiment the present disclosure encompasses the above described cocrystals/salts of baricitinib and solid state forms thereof for use in the preparation of pharmaceutical compositions and/or formulations, preferably for the treatment of rheumatoid arthritis.

In another embodiment the present disclosure encompasses the use of the above described cocrystals/salts of baricitinib and solid state forms thereof for the preparation of pharmaceutical compositions and/or formulations.

The present disclosure further provides pharmaceutical compositions including the cocrystals/salts of baricitinib and solid state forms thereof of the present disclosure.

In yet another embodiment, the present disclosure encompasses pharmaceutical formulations including cocrystals/salts of baricitinib and solid state forms thereof of the present disclosure, and at least one pharmaceutically acceptable excipient.

Pharmaceutical formulations of the present invention contain any one or a combination of the cocrystals/salts of baricitinib and solid state forms thereof of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, the active ingredient and any other solid excipients may be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present disclosure is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

In embodiments, a pharmaceutical formulation of baricitinib is formulated for administration to a mammal, such as a human. Baricitinib acid can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The present disclosure encompasses a process to prepare said formulations of cocrystals/salts of baricitinib and solid state forms thereof by combining the cocrystal prepared according to the present disclosure and at least one pharmaceutically acceptable excipient.

Cocrystals/salts of baricitinib and solid state forms thereof as defined herein, as well as the pharmaceutical compositions or formulations of baricitinib can be used as medicaments, particularly for the treatment of rheumatoid arthritis.

The present disclosure also provides a method of treating of rheumatoid arthritis, by administering a therapeutically effective amount of baricitinib prepared according to the present disclosure, or at least one of the above pharmaceutical compositions or formulations, to a subject suffering from rheumatoid arthritis, or otherwise in need of the treatment.

The present disclosure also provides the use of cocrystals/salts of baricitinib and solid state forms thereof, or at least one of the above pharmaceutical compositions or formulations for the manufacture of a medicament for treating rheumatoid arthritis.

Having described the cocrystals/salts of baricitinib with reference to certain exemplary embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

Powder X-ray diffraction pattern (“PXRD”) method:

Sample is powdered in a mortar and pestle and applied directly on a silicon plate holder. The X-ray powder diffraction pattern was measured with Philips X′Pert PRO X-ray powder diffractometer, equipped with Cu irradiation source=1.54184 \acute{Å} (\acute{Å}ngstrom), X′Celerator (2.022° 20) detector. Scanning parameters: angle range: 3-40 deg., step size 0.0167, time per step 37 seconds, continuous scan.

The described peak positions were determined using silicon powder as an internal standard in an admixture with the sample measured. The position of the silicon (Si) peak was corrected to silicone theoretical peak: 28.45 degrees two theta, and the positions of the measured peaks were corrected respectively.

Solid-State NMR (“ssNMR”) Method

Solid-state NMR spectra were acquired on Agilent Technologies NMR System 600 MHz NMR spectrometer equipped with 3.2 mm NB dual resonance HX MAS probe. NMR spectra of sample SCC-60518-C in solution were recorded on Agilent Technologies 600 MHz NMR spectrometer equipped with 5 mm HCN cryo-probe. Larmor frequencies of proton, carbon and nitrogen nuclei were 599.44, 150.75 and 60.74 MHz, respectively. ¹H and ¹³C NMR chemical shifts are reported relative to TMS (S 0.0 ppm). Samples were at 16,000 (¹H-¹³C CP-MAS).

### Single-Crystal X-Ray Diffraction (“SCXRD”) Method

Diffraction measurements were performed on an Oxford Diffraction Xcalibur Kappa CCD X-ray diffractometer with graphite-monochromated MoKα (λ=0.71073 Å) radiation. The data sets were collected using the ω scan mode over the 20 range up to 54°. Programs CrysAlis CCD and CrysAlis RED were employed for data collection, cell refinement, and data reduction. The structure was solved by direct methods and refined using the SHELXS and SHELXL programs, respectively. The structural refinement was performed on |F|2 using all data. All calculations were performed using the WinGX crystallographic suite of programs.

### EXAMPLES

Baricitinib can be prepared according to the procedure described in U.S. Pat. Nos. 9,938,283 or 8,158,616.

### Example 1: Preparation of Cocrystal of Baricitinib and Orotic Acid Form I

102.0 mg of crude Baricitinib and 47.9 mg of orotic acid hydrate (ratio 1:1) was milled in agate jar with 2 agate balls for 40 minutes at frequency of 25 Hz with 35 μL of Ethanol (absolute). The obtained solid corresponds to form I as confirmed by PXRD.

### Example 2: Preparation of Cocrystal of Baricitinib and Orotic Acid Form II

Crude Baricitinib (50 mg) was dissolved in 1.0 mL of mixture of solvents (2-Propanol:Water=2:1) at 65° C. Orotic acid hydrate (23.4 mg) was dissolved in 3.5 mL mixture of solvents (2-Propanol:Water=2:1) at 50° C. Clear solutions were mixed and left at room temperature to crystallize. Obtained solid was filtered over the white ribbon under the vacuum and corresponds to form II as confirmed by PXRD.

### Example 3: Preparation of Baricitinib Naphthalene-2-Sulfonic Acid Salt Form I

93.0 mg of crude Baricitinib and 56.6 mg of naphthalene-2-sulfonic acid hydrate (ratio 1:1) was milled in agate jar with 2 agate balls for 40 minutes at frequency of 25 Hz with 35 μL of Ethanol (absolute). The obtained solid corresponds to form I as confirmed by PXRD.

### Example 4: Preparation of Cocrystal of Baricitinib and (+)-Camphoric Acid Cocrystal Form I

Baricitinib (99 mg) and (+)-camphoric acid (60 mg) were milled with addition of ethanol 96% (35 μL) in agate jar with 2 agate balls for 330 min at 25 Hz. The obtained solid corresponds to Baricitinib and (+)-camphoric acid cocrystal form I as confirmed by PXRD.

### Example 5: Preparation of Cocrystal of Baricitinib and Orotic Acid Form IV

Baricitinib (99 mg) and orotic acid hydrate (48 mg) were milled with addition of drops of acetone in an agate jar with 2 agate balls for 60 min at 25 Hz. The obtained solid corresponds to Baricitinib and orotic acid cocrystal form IV as confirmed by PXRD.

### Example 6: Preparation of Cocrystal of Baricitinib and Fumaric Acid Form I

Baricitinib was milled in ball mill for 1 hour, using Zirconium oxide jars of 45 ml and 7 balls to obtain Baricitinib amorphous form. Amorphous Baricitinib (62 mg) and fumaric acid (38 mg) were suspended in 1 ml of tetrahydrofuran at room conditions. The obtained solid corresponds to Baricitinib and fumaric acid cocrystal form I as confirmed by PXRD.

### Example 7: Preparation of Cocrystal of Baricitinib and Tartaric Acid Form I

Amorphous Baricitinib (43 mg) and tartaric acid (17 mg) were mixed in Eppendorf tube of 2 ml. Tube was placed into the sauna, that is, a crystallization flask of 6 ml filled with 2 ml of ethanol (96%). The system was left for 7 days at room conditions. The obtained solid corresponds to Baricitinib and tartaric acid cocrystal form I as confirmed by PXRD.

### Example 8: Preparation of Cocrystal of Baricitinib and Tartaric Acid Form II

Amorphous Baricitinib (43 mg) and tartaric acid (17 mg) were mixed in Eppendorf tube of 2 ml. Tube was placed into the sauna, that is, a crystallization flask of 6 ml filled with 2 ml of tetrahydrofuran. The system was left for 7 days at room conditions. The obtained solid corresponds to Baricitinib and tartaric acid cocrystal form II as confirmed by PXRD.

### Example 9: Preparation of Cocrystal of Baricitinib and Succinic Acid Form I

Amorphous Baricitinib (76 mg) and succinic acid (24 mg) were milled in ball-mill with 20 μL of ethanol (96%). Milling was performed at a frequency of 25 Hz for 60 minutes. The obtained solid corresponds to Baricitinib and succinic acid cocrystal form I as confirmed by PXRD.

### Example 10: Preparation of Cocrystal of Baricitinib and Tartaric Acid Form I

Amorphous Baricitinib (43 mg) and L-(+)-tartaric acid (17 mg) were mixed in Eppendorf tube of 2 ml. Tube was placed into the sauna, that is, a crystallization flask of 6 ml filled with 2 ml of ethanol (96%). The system was left for 7 days at room conditions. The obtained solid corresponds to Baricitinib and tartaric acid cocrystal form I as confirmed by PXRD.

### Example 11: Preparation of Cocrystal of Baricitinib and Tartaric Acid Form I

To a solution of 20 grams of Baricitinib in 240 ml of acetone/absolute EtOH/water 3/1/0.5 heated at 60-65° C., a solution of 32.4 grams of L-(+)-tartaric acid in 140 ml of absolute EtOH heated at about 55° C. was added dropwise. The obtained reaction mixture was cooled at 40-45° C. and seeded with 0.1 grams of Baricitinib and L-(+)-tartaric acid co-crystal Form 1. The obtained suspension was stirred at 40-45° C. for about 1 hour, cooled at 20-25° C. and stirred for about 22.5 hours. The crystals were filtered, washed with 2×50 ml of absolute EtOH/acetone 4/1 and dried at 50° C./20 mbar for about 4 hours. 23.6 grams of white crystals of Baricitinib and L-(+)-tartaric acid cocrystal Form 1 were obtained.

### Example 12: Preparation of Single Crystal Baricitinib and Fumaric Acid Co-Crystal Form I

### Preparation of Single Crystal Baricitinib and Fumaric Acid Co-Crystal Form I

10 mg of Baricitinib and fumaric acid co-crystal Form 1 was dissolved in 5 mL of methanol by heating up to 40° C. Solution was left in glass vial covered with parafilm to slowly evaporate. After 7 days, the solvent had evaporated and the material was analyzed by SCXRD. The crystal structure is shown in FIG. 11.

| Space | | | Cell | | |
|---|---|---|---|---|---|
| group | Cell lengths/Å | Cell angles | volume | Z | T/K |
| P2₁/c | a = 6.7554(9) | α = 90 | 2065.25 | 4 | 150 |
| | b = 12.8893(13) | β = 97.528(13) | | | |
| | c = 23.925(3) | γ = 90 | | | |

### Example 13: Preparation of Single Crystal Baricitinib and L-(+)-Tartaric Acid Form I

50 mg of Baricitinib and L-(+)-tartaric acid co-crystal Form 1 was dissolved in 18 mL of Ethyl Acetate by heating up to 40° C. Any residuals were filtered through a Kirsch funnel. Solution was left in glass vial covered with parafilm to evaporate slowly. After 1 month, the solvent had evaporated and the material was analyzed by SCXRD. The crystal structure is shown in FIG. 10.

| Space | | | Cell | | |
|---|---|---|---|---|---|
| group | Cell lengths/Å | Cell angles | volume | Z | T/K |
| P2₁2₁2₁ | a = 5.6685(2) | α = 90 | 2286.79 | 4 | 150 |
| | b = 11.3619(4) | β = 90 | | | |
| | c = 35.5065(15) | γ = 90 | | | |

1. A cocrystal of baricitinib and tartaric acid designated as Form I, characterized by data selected from one or more of the following:
a) a PXRD pattern having peaks at 16.4, 15.6, 10.8, 9.3 and 8.2 degrees 2-theta±0.2 degrees 2-theta
b) an PXRD pattern substantially as depicted in FIG. 7;
c) a solid state ¹³C NMR spectrum having peaks at 147.9, 140.0, 122.7, 114.6, 8.2±0.2 ppm;
d) a solid state ¹³C NMR spectrum having the following chemical shift absolute differences from a reference peak at 8.2 ppm±0.2 ppm of 139.7, 131.8, 114.5 and 106.4±0.1 ppm;
e) a solid state ¹³C NMR spectrum substantially as depicted in FIG. 10;
f) unit cell parameters substantially as follows:
Cell lengths: a=5.6685(2) Å, b=11.3619(4) Å, c=35.5065(15) Å
Cell angles: α=90°, β=90°, γ=90°
Cell volume: 2286.79 Å³; and
Space group P2₁2₁2₁
g) a combination of any two or more of (a), (b), (c), (d), (e) and (f).

2. A cocrystal according to claim 1, characterized by a PXRD pattern having peaks at 16.4, 15.6, 10.8, 9.3 and 8.2 degrees 2-theta±0.2 degrees 2-theta, and also having one, two, three, four or five additional peaks at 24.3, 21.5, 17.6, 16.9 and 15.8 degrees 2-theta 0.2 degrees 2-theta.

3. A cocrystal according to claim 1, wherein the baricitinib and tartaric acid are in a molar ratio of between about 1:0.5 to about 1:2, or about 1:0.8 to about 1:1.1, or about 1:1.

4. A cocrystal according to claim 1, wherein the tartaric acid is L-(+)-tartaric acid.

5. A cocrystal according to claim 1 which is an anhydrous form.

6. A cocrystal according to claim 1, wherein the crystalline form contains 20% or less (w/w) of any solid state forms of baricitinib:tartaric acid cocrystal, or any solid state forms of baricitinib tartrate.

7. A pharmaceutical composition comprising a cocrystal according to claim 1.

8. Use of a cocrystal according to claim 1, the manufacture of a pharmaceutical composition and/or formulation.

9. A pharmaceutical formulation comprising a cocrystal according to claim 1, and at least one pharmaceutically acceptable excipient.

10. A process for preparing a pharmaceutical formulation comprising combining a cocrystal according to claim 1, with at least one pharmaceutically acceptable excipient.

11. A medicament comprising the cocrystal according to claim 1.

12. (canceled)

13. A method for the treatment of rheumatoid arthritis, comprising administering a therapeutically effective amount of a cocrystal according to claim 1, to a subject suffering or otherwise in need of the treatment.

14. A cocrystal according to claim 1, for the manufacture of a medicament for treating rheumatoid arthritis.
